(19) United States
(12) Patent Application Publication
(10) Pub. No.: US 2007/0224142 A1
(43) Pub. Date: Sep. 27, 2007

(54) Hydrogenated castor oil based compositions as a replacement for petrolatum

(76) Inventors: David Frederick Swaile, Cincinnati, OH (US); Barbara Marie Modafari, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/386,925

(22) Filed: Mar. 22, 2006

(51) Int. Cl. A61K 8/37; A61K 36/47

(52) U.S. Cl. 424/66; 424/731

(57) ABSTRACT

An aqueous deodorant composition comprising (a) from about 0.1% to about 89.9% by weight of a hydrogenated castor oil based composition comprising a first oil, said first oil comprising hydrogenated castor oil, and a second oil to soften the hydrogenated castor oil; (b) from about 0.1% to about 30% by weight of a thickening or structuring agent; and (c) from about 10% to about 75% by weight of water.

### FIELD OF THE INVENTION

The present invention relates to aqueous and anhydrous deodorant compositions which are effective at preventing or eliminating malodors resulting from perspiration. In particular, the present invention relates to aqueous and anhydrous deodorant compositions comprising a hydrogenated castor oil based composition composed of a hydrogenated castor oil and a second oil to soften the hydrogenated castor oil. Such a composition functions as a replacement for petrolatum and is especially effective at controlling malodors associated with human perspiration.

### BACKGROUND OF THE INVENTION

Deodorant compositions are well known for use in controlling malodors associated with human perspiration. Malodors develop from human perspiration primarily as the result of microbial interaction with sweat gland secretions which then produces pungent fatty acids. Deodorant compositions typically contain deodorant actives such as antimicrobial agents to help control the microbial development of such malodors and/or they can contain deodorizing fragrances that help to mask the sensory perception of the malodors.

Many deodorant compositions which contain antimicrobial agents and/or fragrances to control or mask malodors resulting from perspiration are typically formulated as deodorant sticks which also contain a thickening agent or other structurant, and a carrier liquid to help solubilize the thickening agent or other structurant. These deodorant formulations are typically applied topically to the underarm or other area of the skin. In addition to being effective at controlling or masking perspiration malodors, these deodorant sticks can provide acceptable aesthetics such as clarity, ease of application, cool and refreshing feel on application, lack of powdery residue, and dry feel.

Many of these deodorant compositions will include an emollient to dissolve the fragrance and control its release rate throughout the day. The emollients can be added by dissolution in the product matrix or by dispersion in the product. The emollients can be of low viscosity and varying polarity such as PPG-3 myristyl ether, PPG-14 butyl ether, tripropylene glycol, dipropylene glycol, and mineral oil. They can also be of high viscosity such as petrolatum, dimethicone gum or polypropylene glycol. These high viscosity materials are sometimes preferred to increase the dry feel and skin substantivity of the product. Unfortunately, such high viscosity materials are generally of very high or very low polarity. Because perfumes are of moderate polarity, such emollients are not always ideal as solvents for perfume raw materials. Therefore, there is a need for high viscosity-moderate polarity materials as solvents for the improvement of fragrance release from aqueous and anhydrous deodorant products.

### SUMMARY OF THE INVENTION

The present invention is directed to aqueous deodorant compositions which comprise (a) from about 0.1% to about 89.9% by weight of a hydrogenated castor oil based composition comprising a first oil, said first oil comprising hydrogenated castor oil, and a second oil to soften the hydrogenated castor oil (b) from about 0.1% to about 30% by weight of a thickening or structuring agent and (c) from about 10% to about 75% by weight of water.

Additionally, the present invention is directed to anhydrous deodorant compositions which comprise (a) from about 0.1% to about 89.9% by weight of a hydrogenated castor oil based composition comprising a first oil, said first oil comprising hydrogenated castor oil, and a second oil to soften the hydrogenated castor oil; (b) from about 0.1% to about 30% by weight of weight of a thickening or structuring agent; and (c) from about 10% to about 90% by weight of a non-aqueous liquid carrier.

It has been found that aqueous and anhydrous deodorant compositions, particularly aqueous deodorant sticks and anhydrous soft deodorant gels or sticks, can be formulated with a hydrogenated castor oil based composition composed of a hydrogenated castor oil and a second softening oil as a replacement for petrolatum. While being extremely mild to the skin, causing little or no skin irritation, such compositions are especially effective at providing improved fragrance longevity.

### DETAILED DESCRIPTION OF THE INVENTION

The present invention relates to aqueous and anhydrous deodorant products that contain a high viscosity-moderately polar emollient comprising a hydrogenated castor oil based composition as a replacement for petrolatum for improved fragrance longevity. Particularly, the present invention provides solid and semi-solid aqueous and anhydrous deodorants comprising a hydrogenated castor oil based composition composed of a hydrogenated castor oil and a secondary oil to soften the hydrogenated castor oil.

While the specification concludes with the claims particularly pointing and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “anhydrous” as used herein means that the deodorant composition of the present invention, and the essential or optional components thereof, are substantially free of added or free water. For example, the deodorant compositions of the present invention may comprise less than about 2%, less than about 1%, less than about 0.5%, or zero percent of free or added water, by weight of the composition.

The term “ambient conditions” as used herein refers to surrounding conditions at about one atmosphere of pressure, 50% relative humidity and about 25° C.

The term “hydrogenated castor oil based composition” includes any of the known hydrogenated castor oil, hydrogenated castor wax or castor wax, i.e. vegetable-based compositions, their derivatives, and mixtures thereof.

The term “volatile” as used herein refers to those materials which have a measurable vapor pressure as measured at 25° C. and 1 atm. Such vapor pressures will typically range from about 0.01 mmHg or from about 0.002 mmHg to about 6 mmHg or to about 1.5 mmHg. Additionally, such materials will have an average boiling point, at about 1 atm, of less than about 250° C. or less than about 235° C. Conversely, the term “non-volatile” refers to those materials which do not have a measurable vapor pressure or which have a vapor pressure of less than about 0.01 mmHg as measured at 25° C. and 1 atm. All viscosity values described herein are measured or determined under ambient conditions, unless otherwise specified.

The solubility parameters for various solvents or other materials described herein are determined by methods well known in the chemical arts. A description of solubility parameters and means for determining them are described by C. D. Vaughan, “Solubility: Effects in Product, Package, Penetration and Preservation”, 103 Cosmetics and Toiletries 47-69, October 1988 and C. D. Vaughan, “Using Solubility Parameters in Cosmetics Formulation”, 36, J. Soc. Cosmetic Chemists 319-333, September/October, 1985.

Hydrogenated Castor Oil Based Composition

Petrolatum is a semi solid mixture of hydrocarbon oils and microcrystalline hydrocarbon wax produced by a variety of methods such as distillation of petroleum oils and blending or “dewaxing” of microcrystalline wax with heavy oils such as mineral oil. Petrolatum is comprised of saturated hydrocarbons and typically does not contain unsaturated materials or materials with polar functional groups, i.e. hydroxyl groups. In contrast, hydrogenated castor oil based compositions are semi-solid mixtures of functionalized and/or non-functionalized hydrocarbon oils blended with hydrogenated castor oil, i.e. castor wax. These materials are a semi-solid like petrolatum but have a generally higher polarity or solubility parameter resulting from the existence of high levels of polar functional groups. Without being bound by theory, it is believed that this higher polarity makes hydrogenated castor oil based compositions more compatible with fragrance components. This increased compatibility is believed to result in a significant reduction of fragrance vapor pressure and therefore result in a perceived fragrance longevity benefit. The oil used to soften the hydrogenated castor wax in the compositions of the present invention may be vegetable-derived or petroleum-derived. Nonetheless, the oil must be of the correct polarity and compatibility with hydrogenated castor oil to produce a high viscosity semi-solid when appropriately blended with hydrogenated castor oil. Furthermore, the softening oil should be a liquid at room temperature.

One example of a hydrogenated castor oil based composition suitable for the present invention is Castorlatum™ available from Chaschem (Bayonne, N.J.). Castorlatum™ is a blend of castor seed oil and hydrogenated castor oil. It is well understood, however, that the castor seed oil may be replaced with a variety of softening oils including, but not limited to, liquid fatty alcohols, such as isostearyl alcohol or octyldodecanol and vegetable oils, such as corn or sun flower oil.

Liquid Carrier

The deodorant compositions of the present invention comprise a liquid carrier suitable for topical application to human skin and appropriate for the product form desired. The liquid carrier is liquid under ambient conditions, and can include one or more liquid carrier materials provided that the any such combination of materials is in liquid form under ambient conditions. Depending on the type of product form desired, concentrations of the liquid carrier in the deodorant compositions will range from about 10% or from about 30% to about 90% or to about 75%, by weight of the deodorant composition.

Liquid carriers suitable for use in the deodorant compositions of the present invention include any topically safe and effective organic, silicone-containing or fluorine-containing, volatile or non-volatile, polar or non-polar liquid carrier, provided that the resulting combination of liquid carrier materials forms a solution or other homogenous liquid or liquid dispersion at the selected processing temperature of the composition. Processing temperatures for the deodorant compositions range from about 50° C. or about 60° C. to about 150° C., to about 120° C. or to about 100° C. Preferred liquid carriers include PPG-3 myristyl ether, propylene glycol, dipropylene glycol, tripropylene glycol, PEG-8, hexylene glycol, glycerin, and mixtures thereof.

Nonlimiting examples of suitable liquid carriers include C₁ to C₂₀ monohydric alcohols, i.e. C₂ to C₈ monohydric alcohols; C₂ to C₄₀ dihydric or polyhydric alcohols, i.e. C₂ to C₂₀ dihydric or polyhydric alcohols; alkyl ethers of all such alcohols, i.e. C₁-C₄ alkyl ethers; and polyalkoxylated glycols, i.e. propylene glycols and polyethylene glycols having from 2 to 30 repeating alkoxylate (e.g., ethoxylate or propoxylate) groups and polyglycerols having from 2 to 16 repeating glycerol moieties; their derivatives and mixtures thereof.

Specific examples of such alcohol liquid carriers include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, and combinations thereof. Other similar but suitable solvents for use as liquid carriers are described, for example, in U.S. Pat. No. 4,781,917, issued to Luebbe et al., Nov. 1, 1998, U.S. Pat. No. 5,643,558, issued to Provancal et al., Jul. 1, 1997, U.S. Pat. No. 4,816,261, issued to Luebbe et al., Mar. 28, 1989 and EP 404 533 A1, published Dec. 27, 1990 by Smith et al.

The deodorant compositions of the present invention may comprise a silicone liquid carrier. The concentration of the silicone liquid carrier may range from about 10% or from about 15% of a silicone liquid carrier, by weight of the composition to about 90% or to about 65% of a silicone liquid carrier, by weight of the composition. The silicone liquid carriers suitable for use herein may include volatile or non-volatile silicones, provided that these silicone materials have the requisite volatility or non-volatility defined herein.

Nonlimiting examples of suitable silicone liquid carriers for use herein include those volatile silicones that are described in Todd et al., “Volatile Silicone Fluids for Cosmetics”, Cosmetics and Toiletries, 91:27-32 (1976). Suitable amongst these volatile silicones are the cyclic silicones having from about 3 or from about 4 to about 7 or to about 6, silicon atoms. Specifically are those which conform to the formula:

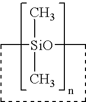

wherein n is from about 3, from about 4 or about 5 to about 7 or to about 6. These volatile cyclic silicones generally have a viscosity value of less than about 10 centistokes. Other suitable silicone liquid carriers for use herein include those volatile and nonvolatile linear silicones which conform to the formula:

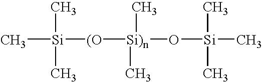

wherein n is greater than or equal to 0. The volatile linear silicone materials will generally have viscosity values of less than 5 cst at 25° C. The non-volatile linear silicone materials will generally have viscosity values of greater than 5 cst at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof.

Specific examples of suitable non-volatile linear silicones for use herein include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone liquid carriers for use in the deodorant compositions of the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistoke to about 200 centistokes or to about 50 centistokes, as measured under ambient conditions.

Other suitable liquid carriers for use in the deodorant compositions of the present invention include, but are not limited to, organic liquid carriers such as mineral oil, petrolatum, isohexadecane, isododecane, various other hydrocarbon oils, and mixtures thereof. Preferred are mineral oil and branched chain hydrocarbons having from about 4 or from about 6 carbon atoms to about 30 or to about 20 carbon atoms. Specific non-limiting examples of suitable branched chain hydrocarbon oils include isoparaffins available from Exxon Chemical Company as Isopar C™ (C7-C8 Isoparaffin), Isopar E™ (C8-C9 Isoparaffin), Isopar G™ (C10-11 Isoparaffin), Isopar H™ (C11-C12 Isoparaffin), Isopar L™ (C11-C13 Isoparaffin), Isopar M™ (C13-C14 Isoparaffin), and combinations thereof. Other nonlimiting examples of suitable branched chain hydrocarbons include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons include petroleum distillates such as those available from Phillips Chemical as Soltrol™ 130, Soltrol™ 170, and those available from Shell as Shell Sol™ 70,-71, and -2033, and mixtures thereof.

Nonlimiting examples of other suitable organic liquid carriers include the Norpar™ series of paraffins available from Exxon Chemical Company as Norpar™ 12, -13, and -15; octyldodecanol; butyl stearate; diisopropyl adipate; dodecane; octane; decane; C₁-C₁₅ alkanes/cycloalkanes available from Exxon as Exxsol™ D80; C₁₂-C₁₅ alkyl benzoates available as Finsolv-TN™ from Finetex; and mixtures thereof. Other suitable liquid carriers include benzoate co-solvents, cinnamate esters, secondary alcohols, benzyl acetate, phenyl alkane, and combinations thereof.

The deodorant compositions of the present invention may be formulated as an aqueous or anhydrous composition. Aqueous deodorant compositions may comprise from about 10% or from about 15% water, by weight of the composition to about 75%, to about 60%, or to about 50% water, by weight of the composition. Anhydrous deodorant compositions may comprise less than about 10%, less than about 3%, less than about 1%, or zero percent water, by weight of the composition.

Thickening or Structuring Agent

The deodorant compositions of the present invention comprise a suitable thickening or structuring agent in order to provide the desired hardness and application characteristics to the compositions. The thickening or structuring agent concentrations may range from about 0.1%, from about 1% or from about 5% of a thickening or structuring agent, by weight of the composition to about 30%, to about 25%, or to about 20% of a thickening or structuring agent, by weight of the composition.

Aqueous deodorant compositions of the present invention may comprise a thickening or structuring agent that can melt to form a solution or other homogenous liquid or liquid dispersion within the liquid carrier at a processing temperature of from about 50° C. or from about 60° C. to about 150° C., to about 120° C. or to about 100° C. Suitable thickening or structuring agents for use in the aqueous deodorant compositions of the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, triglycerides, and other suitable gellants. Other examples include finely divided or colloidal silicas, fumed silicas, and silicates, which includes montmorillonite clays and hydrophobically treated montmorillonites, e.g., bentonites, hectorites and colloidal magnesium silicates.

Anhydrous deodorant compositions of the present invention may comprise any known thickening or structuring agent that provide the deodorant composition with the desired gel matrix and product hardness after formulation and completion of processing. Suitable thickening or structuring agents for use in the anhydrous deodorant compositions of the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, triglycerides, inorganic materials such as clays or silicas, and other suitable non-polymeric gellants.

Preferred thickening or structuring agents for use in the aqueous and anhydrous deodorant compositions are the solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium and potassium salts. For example, suitable salt forming cations may be selected from the group consisting of sodium stearate, sodium palmitate, potassium stearate, potassium palmitate, sodium myristate, aluminum monostearate, and combinations thereof. These thickening or structuring agents may be used at concentrations ranging from about 0.1%, from about 1% or from about 5%, by weight of the composition to about 30%, to about 25%, to about 20%, or to about 10%, by weight of the composition.

Nonlimiting examples of other suitable thickening or structuring agents for use in the aqueous and anhydrous deodorant compositions include fatty alcohols having from about 8 or from about 12 carbon atoms to about 40, to about 30, or to about 18 carbon atoms. Such thickening or structuring agents are wax-like materials which may be included at concentrations ranging from about 1%, from about 5%, or from about 10%, by weight of the composition to about 30% or to about 20%, by weight of the composition. For example, suitable fatty alcohol thickening or structuring agents may be selected from the group consisting of cetyl alcohol, myristyl alcohol, stearyl alcohol, behenyl alcohol, and combinations thereof.

Nonlimiting examples of other suitable thickening or structuring agents for use in the aqueous and anhydrous deodorant compositions include fatty acid esters such as triglycerides. Specific examples of suitable triglyceride thickening or structuring agents include, but are not limited to, tristearin, tribehenin, behenyl palmityl behenyl triglyceride, palmityl stearyl palnityl triglyceride, hydrogenated vegetable oil, hydrogenated rape seed oil, castor wax, fish oils, tripalmitin, Syncrowax HRC™ and Syncrowax HGL-C™ (available from Croda, Inc.). Other suitable glycerides include, but are not limited to, glyceryl stearate and glyceryl distearate. Preferred are glyceryl tribehenin and other triglycerides wherein at least about 75% or about 100% of the esterified fatty acid moieties of the other triglycerides each have from about 18 to about 36 carbon atoms and wherein the molar ratio of glyceryl tribehenin to the other triglycerides is from about 20:1, from about 10:1, or from about 6:1 to about 1:1, to about 3:1, or to about 4:1. The esterified fatty acid moieties may be saturated or unsaturated, substituted or unsubstituted, linear or branched, but are preferably linear, saturated, unsubstituted ester moieties derived from fatty acid materials having from about 18 to about 36 carbon atoms. For example, compositions of the present invention may comprise a triglyceride gelling agent comprising a combination of glyceryl tribehenin and C18-C36 triglyceride.

Nonlimiting examples of other suitable thickening or structuring agents for use in the aqueous and anhydrous deodorant compositions include fatty acids and hydroxy fatty acids such as alpha or beta hydroxy fatty acids having from about 10 to about 40 carbon atoms, and esters and amides of such thickening or structuring agents. Specific nonlimiting examples of such thickening or structuring agents include 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred are 12-hydroxystearic acid, esters of 12-hydroxystearic acid, amides of 12-hydroxystearic acid and combinations thereof. For example, compositions of the present invention may comprise thickening or structuring agents selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof.

Nonlimiting examples of other suitable thickening or structuring agents for use in the aqueous and anhydrous deodorant compositions may be selected from the group consisting of disubstituted or branched monoamide gellants, monosubstituted or branched diamide gellants, triamide gellants, and combinations thereof. For example, such gellants may be selected from the group consisting of n-acyl amino acid derivatives (e.g. n-acyl amino acid amides and n-acyl amino acid esters prepared from glutamic acid), lysine, glutamine, aspartic acid, and combinations thereof. Other suitable amide gelling agents are described in U.S. Pat. No. 5,429,816, issued to Hofrichter et al., Jul. 4, 1995 and U.S. Pat. No. 5,840,287, issued to Guskey et al., Nov. 24, 1998. Concentrations of all such gellants may range from about 0.1%, from about 1%, or from about 5%, by weight of the composition to about 25% or to about 15%, by weight of the composition.

Product Hardness

The deodorant compositions of the present invention may be in the form of a deodorant stick comprising a product hardness of less than about 200 pens (measured in tenths of a millimeter), for example, from about 50 pens or from about 75 pens to about 200 pens or to about 120 pens. As used herein, the term “product hardness” is a reflection of how much force is required to move a penetration needle a specified distance and at a controlled rate into a deodorant composition according to test conditions included herein. Lower values represent harder product and higher values represent softer product. These values can be determined according to the standard procedure set forth by ASTM Method D-5. The product hardness values used herein are measured using an automatic fixed time penetrometer (e.g., Fisher Scientific Co., Model 13-399-10 or equivalent) and a taper-tipped penetration needle as specified in ASTM Method-D 1321-DIN 51 579. The total weight of the needle and shaft in the penetrometer is 50.00.+−.0.05 grams. The deodorant stick compositions are stored at about 26.7° C. (80° F.) for at least 24 hours prior to the determination of the product hardness values of the compositions.

Optional Components

In addition to the aforementioned components, the deodorant compositions of the present invention may further comprise one or more optional components which may modify the physical or chemical characteristics of the compositions or serve as additional “active” components when deposited on the skin. Of course, such optional components may be included provided that they are physically and chemically compatible and do not otherwise unduly impair product stability, aesthetics, or performance. Nonlimiting examples of such optional materials include, but are not limited to, pH buffering agents, additional malodor controlling agents such as deodorant actives, fragrance materials, emollients, humectants, soothing agents, dyes and pigments, medicaments, baking soda and related materials, preservatives, and soothing agents such as aloe vera, allantoin, D-panthenol, avocado oil and other vegetative oils, and lichen extract. Specifically, nonlimiting examples of suitable optional components are described in detail below.

Optional Deodorant Active

The deodorant compositions of the present invention may further comprise a deodorant active to help prevent or eliminate malodors resulting from perspiration. The concentration of the optional deodorant active may range from about 0.001%, from about 0.01%, of from about 0.1%, by weight of the composition to about 20%, to about 10%, to about 5%, or to about 1%, by weight of the composition. Suitable optional deodorant actives may include any topical material that is known or otherwise effective in preventing or eliminating malodor associated with perspiration. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyli amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

Optional Fragrance Materials

The deodorant compositions of the present invention may further comprise one or more fragrance materials to help cover or mask malodors resulting from perspiration or which otherwise provide the compositions with the desired perfume aroma. These optional fragrances may include any perfume or perfume chemical suitable for topical application to the skin.

The concentration of the optional fragrance in the deodorant compositions should be effective to provide the desired aroma characteristics or to mask malodor wherein the malodor is inherently associated with the composition itself or is associated with malodor development from human perspiration. Also, the optional fragrance and whatever carriers accompany it should not impart excessive stinging to the skin, especially broken or irritated skin, at the concentrations disclosed herein. Deodorant compositions of the present invention may comprise optional fragrances selected from the group consisting of free perfumes, encapsulated perfumes, and mixtures thereof.

The optional free perfume for use in the deodorant compositions of the present invention may include one or more individual perfume chemicals provided that the optional free perfume can emit a detectable perfume odor or can mask or help to mask odors associated with perspiration. Generally, the deodorant compositions of the present invention may comprise the optional free perfume at concentrations ranging from about 0.001%, from about 0.1%, or from about 0.5%, by weight of the composition to about 20%, to about 10%, or to about 5%, by weight of the compositions.

Nonlimiting examples of fragrance materials suitable for use as an optional free perfume or an optional encapsulated perfume include any known fragrances in the art or any otherwise effective fragrance materials. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Optional fragrances also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the present invention may comprise optional fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof.

Other suitable optional fragrances are those which mask or help to mask odors associated with perspiration (also referred to herein as odor masking fragrances), some non-limiting examples of which are described in U.S. Pat. No. 5,554,588, issued to Behan et al., Sep. 10, 1996, U.S. Pat. No. 4,278,658, issued to Hooper et al., Dec. 8, 1981, U.S. Pat. No. 5,501,805, issued to Behan et al., Mar. 26, 1996, and EP Patent Application 684 037 A1, published Nov. 29, 1995, by Gordon et al. Preferred optional odor masking fragrances are those which have a Deodorant Value of from about 0.25 or from about 0.9 to about 3.5, as measured by the Deodorant Value Test described in EP Patent Application 684 037 A1, by Gordon et al., published Nov. 29, 1995. Optional fragrances of the present invention may also comprise solubilizers, diluents, or solvents which are well known in the art. Such materials are described in Arctander, Perfume and Flavour Chemicals (Aroma Chemicals), Vol. I and II (1969). These materials typically include small amounts of dipropylene glycol, diethylene glycol, C₁-C₆ alcohols, and/or benzyl alcohol.

Method of Manufacture

The deodorant compositions of the present invention may be prepared by any known or otherwise effective technique suitable for providing a deodorant composition as described herein. Methods for preparing the deodorant compositions of the present invention include conventional formulation and mixing techniques. For example, one suitable method combines the liquid carrier and the gellant. The mixture is then heated with agitation to a temperature of from about 75° C. to about 150° C. to allow the gellant to melt. The resulting solution is cooled before adding a mixture of fragrance and the hydrogenated castor oil based composition of the present invention. The cooled composition is then poured into an appropriate container or dispenser at about 70° C. and allowed to solidify within the container or dispenser by cooling or allowing to cool the contained composition to ambient temperature.

Method of Use

The deodorant compositions of the present invention may be topically applied to the axilla or other area of the skin in any known or otherwise effective method for controlling malodor associated with perspiration. These methods comprise applying to the axilla or other area of the human skin a safe and effective amount of the deodorant composition of the present invention. “Safe and effective amount” means an amount of the deodorant composition topically applied to the skin which is effective in inhibiting or minimizing or masking perspiration malodor at the site of application while also being safe for human use at a reasonable risk/benefit ratio. Thus, a safe and effective amount, as used in the present invention may range from about 0.1 gram per axilla to about 2.0 gram per axilla. The compositions are preferably applied to the axilla or other area of the skin one or more times daily, preferably once daily.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention. All exemplified concentrations are weight-weight percents, unless otherwise specified. Each of the exemplified deodorant stick compositions may be applied topically to the underarm in an amount ranging from about 0.1 gram to about 2 grams per axilla. The compositions are effective in reducing, masking or eliminating perspiration odor, and are mild to the skin causing little or no skin irritation.

### Examples I-II

Example 1 is an aqueous deodorant product that is formulated by combining all ingredients except the fragrance and castorlatum™ in container 1 and then heating the mixture until it forms a clear solution at approximately 80° C. In a separate container, (container 2) the castorlatum™ is heated to about 55° C. to reduce its viscosity and then the fragrance is added to the castorlatum™. Container 1 is then cooled to approximately 75° C. and the contents of container 2 are added to container 1 under strong agitation to assure proper dispersion of the castorlatum™ phase. The resulting mixture is then poured into an appropriate dispenser or other container and allowed to solidify by cooling to ambient temperature. Hardness measurement of this product shows approximately 95 pens.

Example 2 is an anhydrous deodorant product that is formulated by combining all of the liquid ingredients in container 1 and then adding the structuring agents (stearyl alcohol, hydrogenated castor oil, and behenyl alcohol). This mixture is then heated at about 85° C. until a clear solution is formed. The powder components of the formula are then added. In a separate container (container 2) the castorlatum™ is heated to about 55° C. to reduce its viscosity and then the fragrance is added to the castorlatumr™. Container 1 is then cooled to approximately 70° C. and the contents of container 2 are added to container 1 under agitation to assure proper dispersion of the castorlatum™ phase. The resulting mixture is then cooled to about 58° C. and poured into an appropriate dispenser or other container and allowed to solidify by cooling to ambient temperature

TABLE 1
Example formulas

| | Example I | Example II |
|---|---|---|
| Component | (wt %) | (wt %) |
| Water | 22.0 | |
| Dipropylene glycol | 50.0 | |
| Propylene glycol | 18.0 | |
| Sodium stearate | 5.5 | |
| Castorlatum ™ | 2.0 | 1.0 |
| Fragrance | 2.5 | 1.0 |
| Aluminum zirconium | | 24.0 |
| Trichlorohydrex gly | | |
| Cyclopentasiloxane | | 40.8 |
| Stearyl alcohol | | 16.0 |
| Phenyl trimethicone | | 8.5 |
| Hydrogenated castor oil | | 4.75 |
| PEG-8 distearate | | 2.0 |
| Mineral oil | | 1.5 |
| Silica | | 0.25 |
| Behenyl alcohol | | 0.2 |

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the term in a document incorporated herein by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous deodorant composition comprising (a) from about 0.1% to about 89.9% by weight of a hydrogenated castor oil based composition comprising a first oil, said first oil comprising hydrogenated castor oil, and a second oil to soften the hydrogenated castor oil; (b) from about 0.1% to about 30% by weight of a thickening or structuring agent; and (c) from about 10% to about 75% by weight of water.

2. The aqueous deodorant composition of claim 1 wherein said second oil is selected form the group consisting of castor seed oil, liquid fatty alcohols, vegetable oils, and mixtures thereof.

3. The aqueous deodorant composition of claim 1 wherein said thickening or structuring agent is selected from the group consisting of fatty acid gellants, salts of fatty acids, hydroxy fatty acid gellants, esters and amides of fatty acids, chloesterolic materials, dibenzylidene alditols, lanolinolic materials, fatty alcohols, triglycerides, and mixtures thereof.

4. The aqueous deodorant composition of claim 1 wherein said deodorant composition has a product hardness of less than about 200 pens.

5. The aqueous deodorant composition of claim 1 further comprising from about 0.001% to about 20% of a deodorant active.

6. An anhydrous deodorant composition comprising (a) from about 0.1% to about 89.9% by weight of a hydrogenated castor oil based composition comprising a first oil, said first oil comprising hydrogenated castor oil, and a second oil to soften the hydrogenated castor oil; (b) from about 0.1% to about 30% by weight of weight of a thickening or structuring agent; and (c) from about 10% to about 90% by weight of a non-aqueous liquid carrier.

7. The anhydrous deodorant composition of claim 6 wherein said second oil is selected form the group consisting of castor seed oil, liquid fatty alcohols, vegetable oils, and mixtures thereof.

8. The anhydrous deodorant composition of claim 6 wherein said thickening or structuring agent is selected from the group consisting of fatty acid gellants, salts of fatty acids, hydroxy fatty acid gellants, esters and amides of fatty acids, chloesterolic materials, dibenzylidene alditols, lanolinolic materials, fatty alcohols, triglycerides, and mixtures thereof.

9. The anhydrous deodorant composition of claim 6 wherein said non-aqueous liquid carrier is selected from the group consisting of alcohol liquid carriers, silicone liquid carriers, mineral oil, petrolatum, isohexadecane, isododecane, hydrocarbon oils, and mixtures thereof.

10. The anhydrous deodorant composition of claim 6 wherein said deodorant composition has a product hardness of less than about 200 pens.

11. The anhydrous deodorant composition of claim 6 further comprising from about 0.001% to about 20% of a deodorant active.
